(19) United States
(12) Patent Application Publication
(10) Pub. No.: US 2018/0207026 A1
(43) Pub. Date: Jul. 26, 2018

(54) CONFORMAL THERMAL DEVICE

(71) Applicant: INNOVATION LAB, Newport Beach, CA (US)

(72) Inventor: Scott E. Parazynski, Newport Beach, CA (US)

(21) Appl. No.: 15/747,011

(22) PCT Filed: Jul. 22, 2016
(86) PCT No.: PCT/US16/43737

§ 371 Date: Jan. 23, 2018

Related U.S. Application Data
(60) Provisional application No. 62/196,283, filed on Jul. 23, 2015.

(51) Int. Cl. A61F 7/02; A61B 5/01

(52) U.S. Cl. CPC A61F 7/02; A61F 2007/0096; A61B 5/01

(57) ABSTRACT

A conformal thermal device is described that can conform to one or more body parts of a user for either warming or cooling the body parts to within a desired or therapeutic temperature range. Some embodiments of the conformal thermal device include features that indicate a cooling and/or heating temperature. In addition, some embodiments of the conformal thermal device include modular extensions for adapting the shape of the conformal thermal device to a part of the body. Various other features and functions associated with the conformal thermal device are described herein.

### CROSS-REFERENCE TO RELATED APPLICATION

The current application claims priority to U.S. Provisional patent application Ser. No. 62/196,283, filed on Jul. 23, 2015 and entitled “Conformal Joint Thermal System,” which is incorporated by reference herein in its entirety.

### TECHNICAL FIELD

The subject matter described herein relates to various embodiments of a device that conforms to a body part of a user and provides cooling or heating to the body part.

### BACKGROUND

Some commercially available thermal packs, such as ice packs and heating packs, are available in inconvenient or ineffective shapes for use in treating post-surgical or traumatized body parts and/or joints, e.g., knees, shoulders, ankles, elbows, wrists, spine, and hips. This can result in poor cold or heat transfer to the body part, requiring frequent repositioning of the thermal pack in order to effectively treat the body part, and inadequate therapeutic benefits realized by the body part. Other problems that can arise with some commercial thermal packs include movement restrictions by the user of the thermal pack (e.g., due to the size and/or shape of the thermal pack), discomfort caused by straps that couple the thermal pack to the body part, and/or condensation dampness occurring next to the thermal pack. Although thermal packs can provide therapeutic benefits (e.g., reduction of inflammation and/or pain relief), users of thermal packs may not receive a benefit and can even be harmed from their use (e.g., freezer or heat burns).

### SUMMARY

Disclosed herein include embodiments of a conformal thermal device that is configured to conform to one or more body parts of a user for providing thermal therapy to the body parts. In one aspect, a conformal thermal device is described that includes an outer skin that defines a body of the conformal thermal device. The outer skin can be flexible and configured to conform to a body part of a user. The conformal thermal device can further include an inner chamber defined by and enclosed within the outer skin and a thermal material that is configured to provide cooling or heating to the body part, the thermal material being contained within the inner chamber.

In some variations one or more of the following features can optionally be included in any feasible combination. The body can include one or more of a ring shape, a helical shape, and a torus shape. The body can further include one or more cutouts that extend through a disc-shaped body and are radially positioned about a center of the disc-shaped body. The conformal thermal device can include an adjustable coupling mechanism that extends from the outer skin and is configured to releasably couple the body of the conformal thermal device to the body part such that the body part is able to perform unimpeded movements. The body can include one or more heat seals that define at least two sub chambers that are in fluid communication with each other and allow the thermal material to flow therebetween. The conformal thermal device can include one or more of an elastic support and a compressible support positioned within the body and configured to expand and/or compress in response to movement of the body part. The conformal thermal device can include a first indicator that is configured to indicate when at least one of the thermal material, an outer surface of the outer skin, and a top surface of the user is at a temperature within a defined temperature range. The first indicator can be configured to change color to inform the user that the temperature is within the desired temperature range. The defined temperature range can include approximately negative five degrees Celsius to approximately 5 degrees Celsius, such as for providing cooling, and approximately 18 degrees Celsius to approximately 38 degrees Celsius, such as for providing heating. Various other temperature ranges have been contemplated. The conformal thermal device can include a second indicator that is configured to inform the user when the outer skin has been conformed to the body part for a duration that is within a defined time range. The second indicator can be configured to change color to inform the user that the duration is within the defined time range. The defined time range includes a time range of approximately 15 minutes to approximately 30 minutes. Some defined time ranges can include approximately 19 to approximately 21 minutes. Various other time ranges have been contemplated. The conformal thermal device can include an adjustable weight that is at least one of coupled to and integrated with the outer skin, wherein the adjustable weight is configured to provide adjustable compression between the outer skin and the body part. The body of the conformal thermal device can be pleated and the conformal thermal device can include a cover that is configured to extend around a part of the body and includes an insulation material that limits the release of heat away from the body part. The conformal thermal device can include a cover that is configured to extend around a part of the body and includes a moisture-wicking material that collects moisture released from and adjacent to the body. The conformal thermal device can include at least one modular extension that includes thermal material and is configured to provide cooling or heating, each of the modular extensions being configured to releasably couple to the body of the conformal thermal device. The conformal thermal device can include at least one connector along the outer skin, each of the connectors being configured to releasably couple one or more of the least one modular extension.

In another interrelated aspect of the current subject matter, a method includes conforming an outer skin of a conformal thermal device against a body part of a user, the conformal thermal device can include a thermal material that is configured to provide heating or cooling to the body part. The method can further include providing heating or cooling to the body part.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates an embodiment of a conformal thermal device having a ring or torus shape that encircles a center part;

FIG. 2 illustrates another embodiment of the conformal thermal device having a ring or torus shape with one or more elastic or compressible supports;

FIG. 3 illustrates another embodiment of the conformal thermal device including one or more extensions that assist with stabilizing a position of the conformal thermal device relative to a body part;

FIG. 4 illustrates another embodiment of the conformal thermal device including an elastic feature for allowing the body of the conformal thermal device to expand radially;

FIG. 5A illustrates another embodiment of the conformal thermal device having a helical-shaped body;

FIG. 5B illustrates the conformal thermal device of FIG. 5A positioned on a bent knee joint of the user with a second end of the helical-shaped body positioned on the knee cap of the user;

FIG. 5C illustrates the conformal thermal device of FIG. 5A positioned on a straight knee joint of the user with the second end of the helical-shaped body positioned on the knee cap of the user;

FIG. 6A illustrates another embodiment of the conformal thermal device having a plurality of sub-chambers that are in fluid communication with each other;

FIG. 6B illustrates another embodiment of the conformal thermal device including a plurality of sub-chambers that are in fluid communication with each other, an inner chamber that is enclosed, and at least one indicator;

FIG. 7 illustrates another embodiment of the conformal thermal device having an adjustable coupling mechanism;

FIG. 8A-8B shows perspective views of a flexible cover that is configured to cover an outer surface of the conformal thermal device and provide either heat insulation or moisture wicking properties; and

FIG. 9 illustrates another embodiment of the conformal thermal device that includes one or more pleats.

When practical, similar reference numbers denote similar structures, features, or elements.

### DETAILED DESCRIPTION

Disclosed herein include embodiments of a conformal thermal device that can conform to one or more body parts of a user for either warming or cooling the body parts to within a desired or therapeutic temperature range. Such therapeutic temperature range can include temperatures that provide healing and/or benefits to the body parts. For example, applying a conformal thermal device that has a temperature of approximately 0 degrees to approximately 10 degrees Celsius to an injured joint can assist with healing the injured joint (e.g., reduce swelling, reduce pain, and allow mobility). In addition, in order to achieve therapeutic benefits, it can be necessary to heat or cool the body part for a specific amount of time or within a specified time frame. For example, therapeutic benefits can be lessened or not achieved if cooling of the body part is either not long enough or too long, even if the cooling temperature is within the therapeutic temperature range. Some embodiments of the conformal thermal device include shapes and features that improve the conforming of the device against one or more body parts. This can assist with effective and efficient transfer of heat between the conformal thermal device and a body part, thus allowing the body part to reach a therapeutic temperature. Some embodiments of the conformal thermal device can include modular extensions that can be releasably coupled to and removed from a main body to create a variety of shaped and sized conformal thermal devices. This can allow the conformal thermal device to adapt and provide therapy to a variety of body parts. The conformal thermal device described herein includes a variety of features and includes several benefits for providing thermal therapy (e.g., heating, cooling) to body parts, as will be described in greater detail below.

As described herein, a body part can include one or more body parts or anatomies of a person or animal, and such body parts can include a surface (e.g., skin) area, soft tissue (e.g., muscle, tendon), and/or bone without departing from the scope of this disclosure. Furthermore, the thermal conformal device embodiments and features described herein can be used for either heating or cooling of the body parts.

Some embodiments of the conformal thermal device include an outer skin that defines a main body of the conformal thermal device. The outer skin can be flexible to allow the main body to flex and conform to a body part to which the conformal thermal device is placed in contact with. The main body can include at least one inner chamber and/or sub-chambers that can contain a thermal material. A sub-chamber, as described herein, includes a compartment within the body that is in fluid communication with another compartment (e.g., another sub-chamber) within the body. As such, thermal material can flow between sub-chambers whereas thermal material is confined within an inner chamber. The thermal material is configured to assist with the cooling or heating of the body part, as will be described in greater detail below.

The outer skin can be made out of a variety of materials, such as materials that are waterproof and/or control (e.g., facilitate, prevent) heat transfer. For example, the thermal material can be cooled or have cooling properties for assisting with cooling the body part the conformal thermal device is placed on or adjacent to. The outer skin can allow heat to be transferred from the body part to the conformal thermal device, including transferring at least some of such heat to the thermal material. In addition, for example, the thermal material can be heated or have heating properties for assisting with heating the body part. The outer skin can thus allow heat to be transferred from the conformal thermal device to the body part for efficiently and effectively heating the body part.

The main body can include a variety of features that assist the conformal thermal device with conforming to the adjacent body part. For example, some conformal thermal devices include an outer skin that is expandable to allow the conformal thermal device to expand and contract in one or more directions, such as during movement of the adjacent body part or during positioning of the device. Such features can thus assist with allowing the conformal thermal device to effectively conform to the body part, while also preventing or restricting interference of the functioning and/or movement of the body part. Other features for assisting the conformal thermal device with conforming to body parts can include an elastic or compressible support (e.g., spring) that can elastically deform to allow the device to conform to the adjacent body part while maintaining a desired structure. For example, some compressible supports, such as a spring, can be made out of a material (e.g., metal, stainless steel, etc.) that includes a greater thermal mass than the other components of the device such that the compressible supports can assist with maintaining the desired thermal temperature. Some embodiments of the conformal thermal device include one or more seams, slits, and/or cutouts along the main body of the conformal thermal device can also assist with allowing the device to conform to the adjacent body part. Such features also provide benefits from a manufacturing standpoint as they are relatively easy and cost effective to include in the conformal thermal device.

The thermal material can include one or more of a variety of materials, including Propylene Glycol, Ammonium Nitrate, and/or any conformable material that can provide either heating or cooling. The thermal material can fill an inner chamber or sub-chamber to any varying amount. For example, the thermal material can fill approximately 25%, 50%, 75%, and/or 100% of the inner chamber. Some conformal thermal devices include more than one inner chamber and each inner chamber can be filled with one or more materials (e.g., thermal materials) to any fill amount. For example, one inner chamber can be filled with thermal material to approximately 75% and a second inner chamber can be filled to approximately 50%. Furthermore, the fill volume of material in any one of the sub-chambers can vary, such as according to the conforming needs of the conformal thermal device. For example, at least two sub-chambers can be in fluid communication with each other thereby allowing material (e.g., thermal material) to be transferred therebetween. The material in the sub-chambers can thus move to accommodate the conformal thermal device with conforming to various body parts. Some implementations of the conformal thermal device include at least one inner chamber that is enclosed and prevents movement of the thermal material therefrom. This can be beneficial for ensuring that thermal material is placed adjacent a part of the body that can benefit from either cooling or heating. The thermal material can include a variety of viscosities, including different viscosities at various temperatures. As such, in some embodiments the thermal material can have a greater viscosity as the thermal material cools, or vice-versa.

The main body of the conformal thermal device can include any number of shapes and sizes for effectively conforming to any number of body parts of a user. For example, the body can be ring-shaped or torus-shaped to assist with encircling an injured joint, such as the knee joint. Some embodiments of the main body can include features that assist with providing cooling or heating to specific parts of the body, such as the knee cap or elbow. For example, the main body can include an extension and/or at least partially enclosed inner sub-chamber that is shaped and positioned relative to the rest of the main body such that the extension effectively heats or cools the specific body part when the device is placed on or adjacent such body part.

Some implementations of the conformal thermal device can include one or more indicators that are configured to indicate either a temperature or a time associated with the heating or cooling provided by the conformal thermal device. For example, a temperature indicator can indicate when at least the surface temperature of the conformal thermal device is within a desired temperature range or at a desired temperature. For example, the desired temperature range can include approximately negative five degrees Celsius to approximately 5 degrees Celsius, such as for providing cooling, and approximately 18 degrees Celsius to approximately 38 degrees Celsius, such as for providing heating. Various other temperature ranges have been contemplated. A time indicator can indicate how long the conformal thermal device has been in contact with a body part and/or whether the conformal thermal device has been in contact with the body part for a desired amount of time. For example, the desired amount of time can include a time range of approximately 15 minutes to approximately 30 minutes. Some desired time ranges can include approximately 19 to approximately 21 minutes. Various other time ranges have been contemplated. Such indicators can provide, for example, their respective information in a variety of ways. For example, some indicators can change color to provide either the desired temperature range or indicate the desired time has been achieved. Some embodiments of the indicator can include audio and/or tactile features that provide information to a user regarding temperature and/or time. Any number of visual, tactile, and/or audio features can be included in one or more indicators associated with eh conformal thermal device for indicating temperature and/or time, as discussed above.

Some implementations of the conformal thermal device can include a main body having one or more coupling features or connectors for releasably coupling various sized and shaped modular extensions to the main body. Such modularity can allow the conformal thermal device to be sized and shaped to effectively conform to a variety of body parts for efficiently heating or cooling the body parts.

Some implementations of the conformal thermal device can include an adjustable weight that is either coupled to or integrated with the body of the device. The adjustable weight can be configured to provide adjustable compression between the body and the body part of the user. For example, the body of the conformal thermal device can include a compliant metal that can be formed and shaped to allow the outer surface of the body to conform to the body parts of the user thus providing various compressions against the body part. Such compliant material can also be a cold or heat sink to provide additional cooling or heating properties.

Some implementations of the conformal thermal device include one or more adjustable coupling features that are configured to assist with securing a position of the conformal thermal device relative to the user, such as the one or more body parts in need of thermal therapy (e.g., heating or cooling). The adjustable coupling features, along with the main body and any modular extensions, are configured to allow articulation and/or movement of the body parts to which the conformal thermal device is in contact with and/or adjacent to.

Some implementations of the conformal thermal device can include one or more features that can provide heat insulation and/or moisture wicking properties. For example, an insulator can be coupled to a part of the main body of the conformal thermal device for preventing or reducing heat loss from the main body that is not directed to the body part of the user. As such, this can increase the efficiency and effectiveness of the conformal thermal device heating the body part. In addition, a moisture wicking cover can be coupled to a part of the main body of the conformal thermal device for capturing moisture or condensation that can be released from the conformal thermal device. The wicking cover can thus reduce or eliminate moisture transferring to the user's skin or clothes.

Various embodiments of the conformal thermal device are described herein, of which any can include one or more of the features described herein.

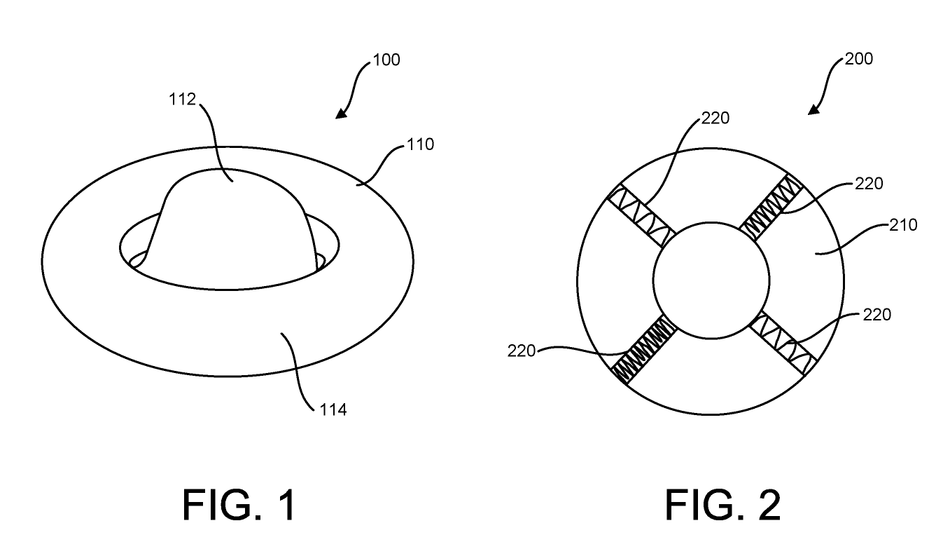

FIG. 1 illustrates an embodiment of a conformal thermal device 100 having a body 110 that is ring-shaped or torus-shaped. The body can further include a center part 112 that is centrally located to the ring-shaped body 110. The conformal thermal device 100 can include at least one inner chamber and/or sub-chamber enclosed within the outer skin 114. In addition, a thermal material can be contained within the inner chamber and/or sub-chamber. As described above, the thermal material is configured to assist with providing thermal therapy to a user, such as by heating or cooling a body part of a user. The ring-shaped portion of the body 110 can include a first inner chamber or sub-chamber and the center part 112 can include a second inner chamber or sub-chamber. As such, thermal material can be contained within or flow between the ring-shaped portion and the center part 112.

For example, the ring-shape of the body 110 can assist with positioning the conformal thermal device 100 on a knee joint such that the ring-shaped portion encircles a part of the knee and the center part 112 can be positioned on or adjacent to the knee cap. This configuration can thus be beneficial for efficiently and effectively cooling or heating the knee joint. In some embodiments, the center part 112 includes an inner chamber that contains thermal material such that a sufficient amount of thermal material is positioned adjacent or on the knee cap when the conformal thermal device 100 is positioned on or adjacent the knee joint. Any of the features described in this disclosure (e.g., indicators, adjustable coupling features, etc.) can also be included in the conformal thermal device 100 or any of the other conformal thermal device embodiments shown or described herein.

FIG. 2 illustrates another embodiment of the conformal thermal device 200 having a body 210 that is ring-shaped or torus-shaped. The conformal thermal device 100 can include an outer skin 114 with at least one inner chamber and/or sub-chamber enclosed within the outer skin 114 with a thermal material contained therein. In addition to any of the features described herein, the conformal thermal device 200 shown in FIG. 2 includes a plurality of elastic or compressible supports 220. Although shown in FIG. 2 as having four elastic supports 220 spaced approximately evenly about the body in a radial direction, the conformal thermal device 200 can include any number of elastic supports 220 positioned in any number of orientations relative to the body 210 and each other. Furthermore, although the elastic supports 220 are shown as a coil or spring, any number of deformable and reformable structures can be included for allowing the conformal thermal device 200 to effectively conform to a variety of body parts of the user. In some implementations, any one of the elastic supports 220 can be made out of a material (e.g., metal material) that acts as a cold or heat sink, thereby assisting with maintaining the therapeutic temperature.

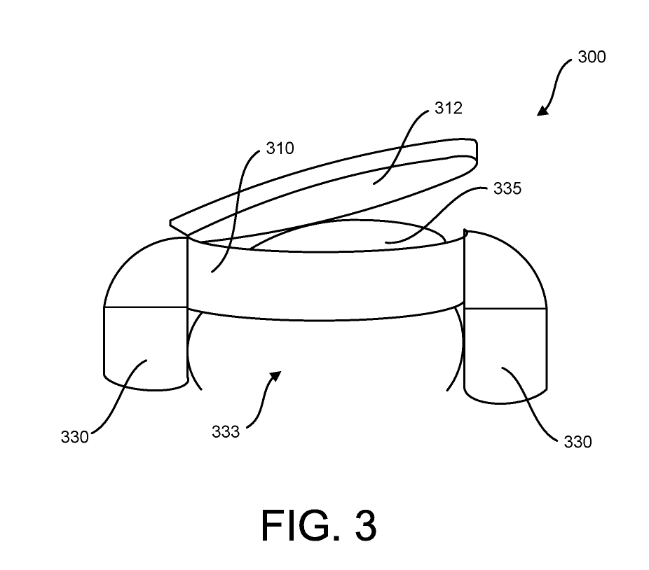

FIG. 3 illustrates another embodiment of the conformal thermal device 300 having a body 310 that is ring-shaped or torus-shaped including one or more support extensions 330 that assist with stabilizing a position of the conformal thermal device 300 relative to a body part. The body 310 can further include a center part 312 that is centrally located to the ring-shaped body 310. Similar to the conformal thermal device 300 of FIG. 1, the ring-shaped portion of the body 310 can include a first inner chamber or sub-chamber and the center part 112 can include a second inner chamber or sub-chamber. As such, thermal material can be contained within or flow between the ring-shaped portion and the center part 112. Furthermore, each of the support extensions 330 can include a third inner chamber or sub-chamber such that the thermal material and/or other material can be at least partially contained therein. For example, at least one support extension 330 can include a weighted material (e.g., heavier than the thermal material, a metal material, and/or etc.) that assists with securing a position of the conformal thermal device 300 around a part of a joint 333 (e.g., knee joint) with the center part 312 positioned on top of the joint 333 (e.g., knee cap 335), as shown in FIG. 3. For example, at least one support extension 330 can include a formable material that assists with forming the body of the device to the adjacent body part. The formable material can also be a cold or heat sink for assisting with maintaining the thermal temperature.

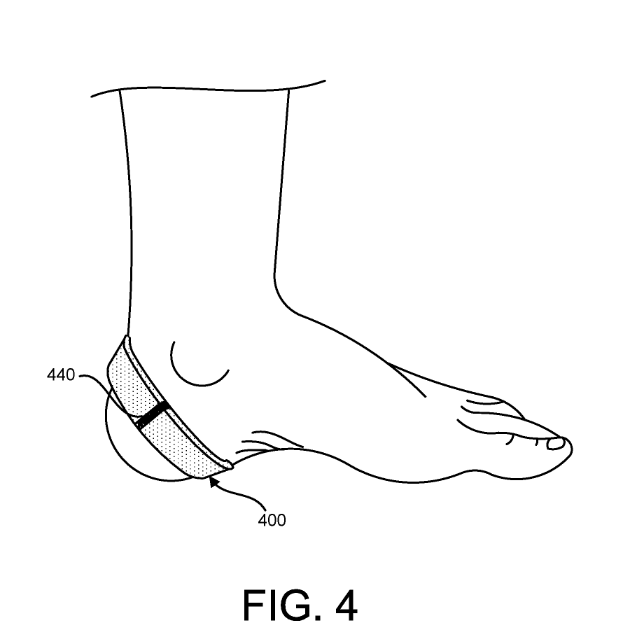

FIG. 4 illustrates another embodiment of the conformal thermal device 400 having a ring-shaped body that is configured to be positioned around a part of the body, such as the heel of a foot. In addition to the any of the features described herein that can be included in the conformal thermal device, the conformal thermal device 400 shown in FIG. 4 includes an elastic feature 440 that can allow the ring-shaped body 410 to radially expand and contract. For example, the elastic feature 440 can include a plurality of pleats that can be expanded to allow the conformal thermal device 400 to be positioned and then contract to secure the position of the conformal thermal device 400 relative to a body part.

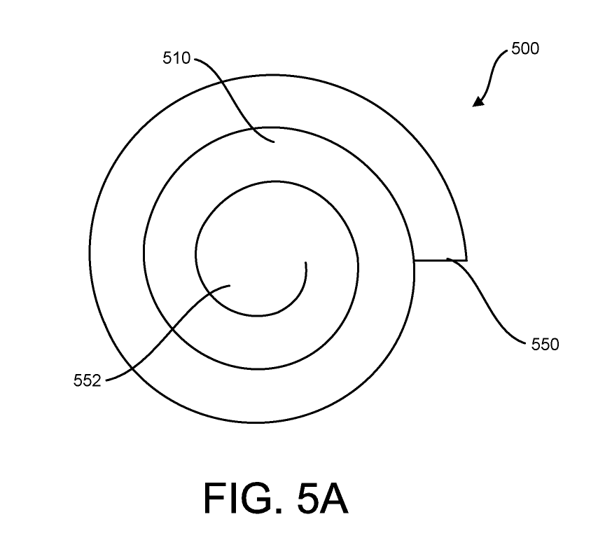

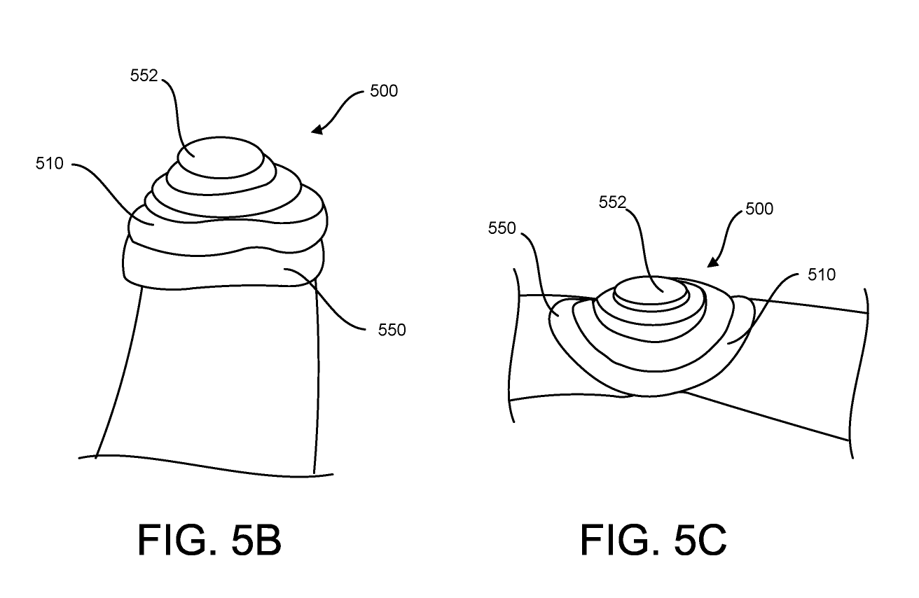

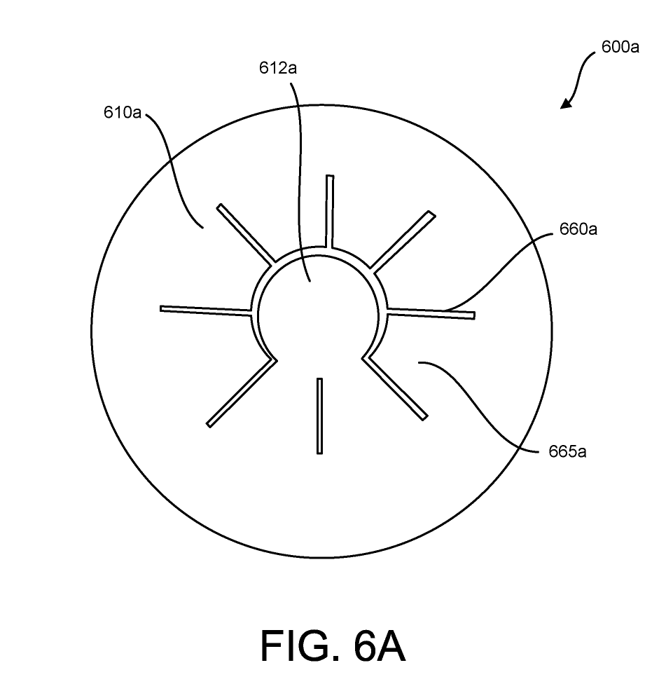

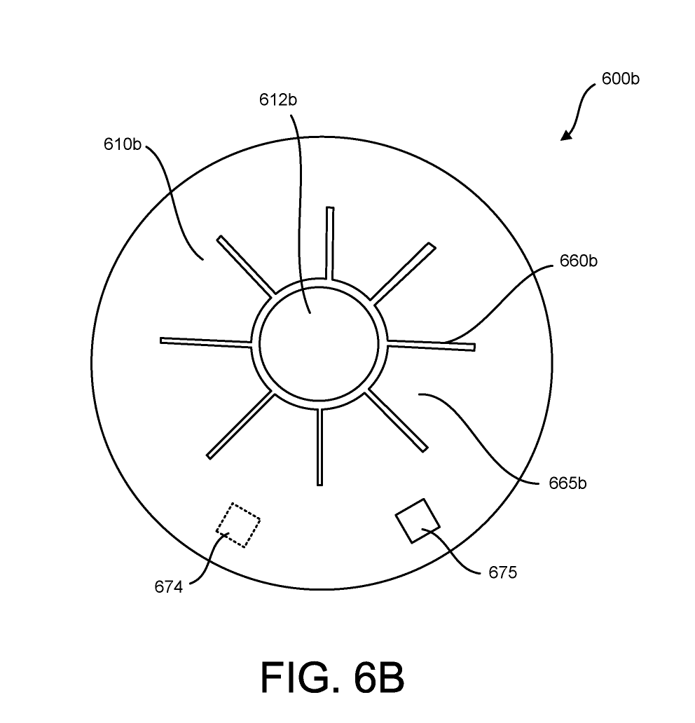

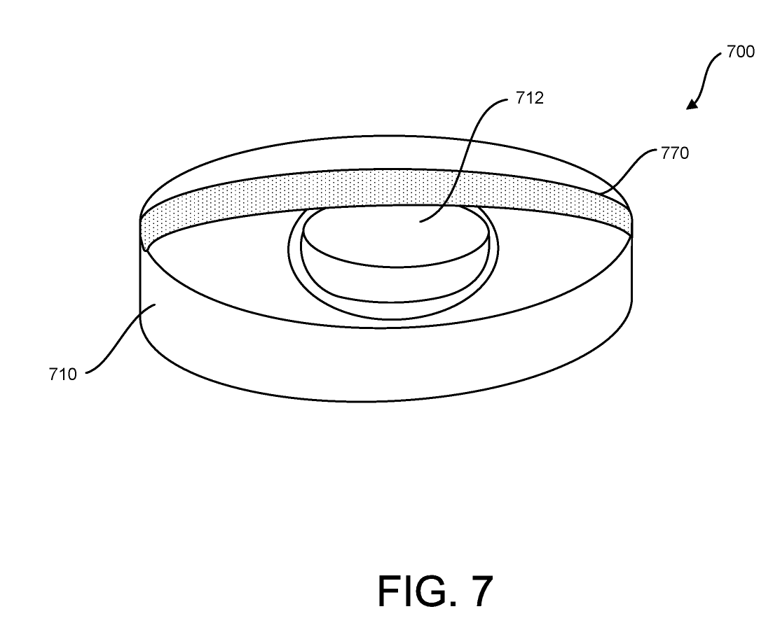

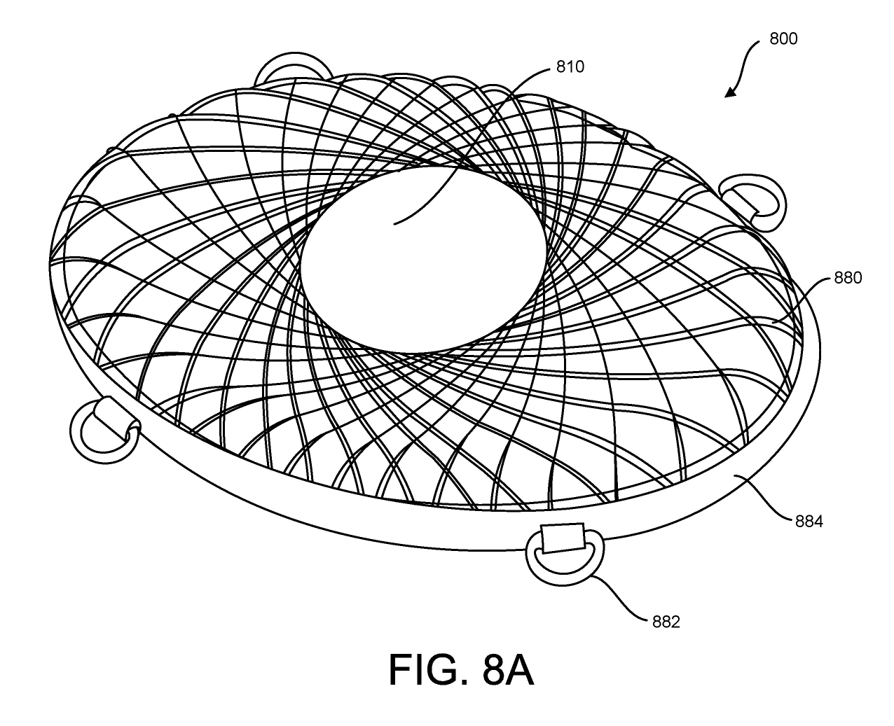

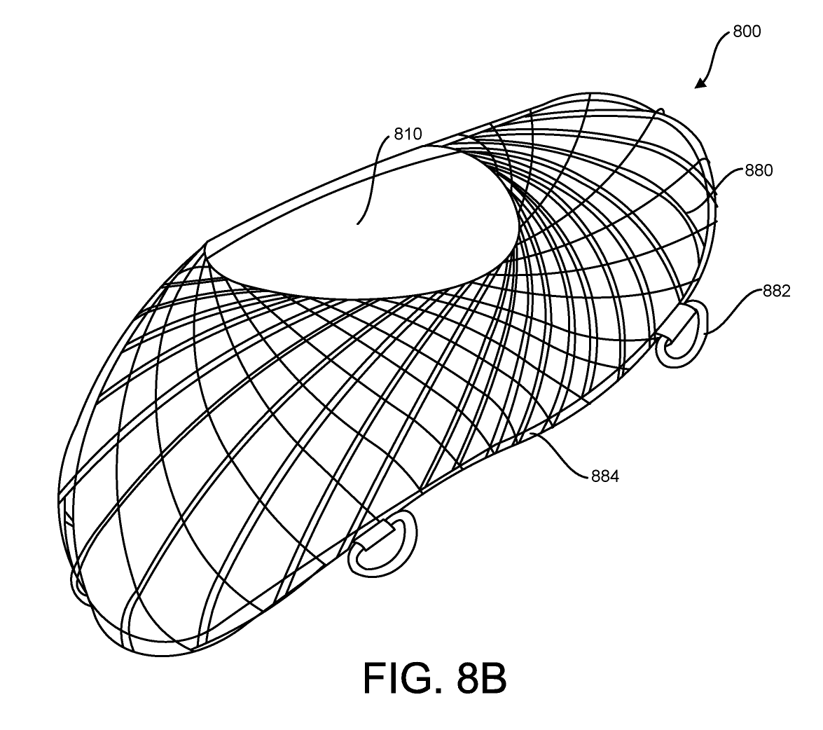

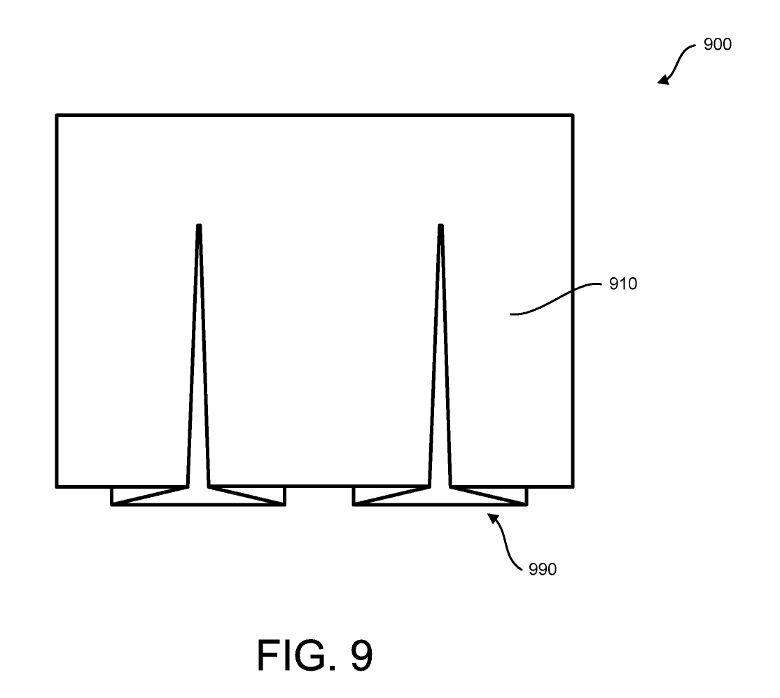

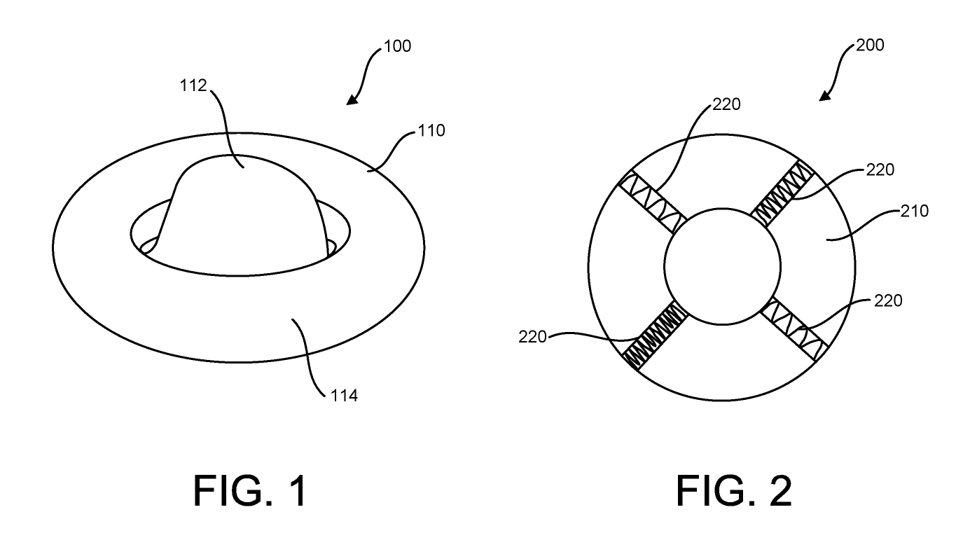

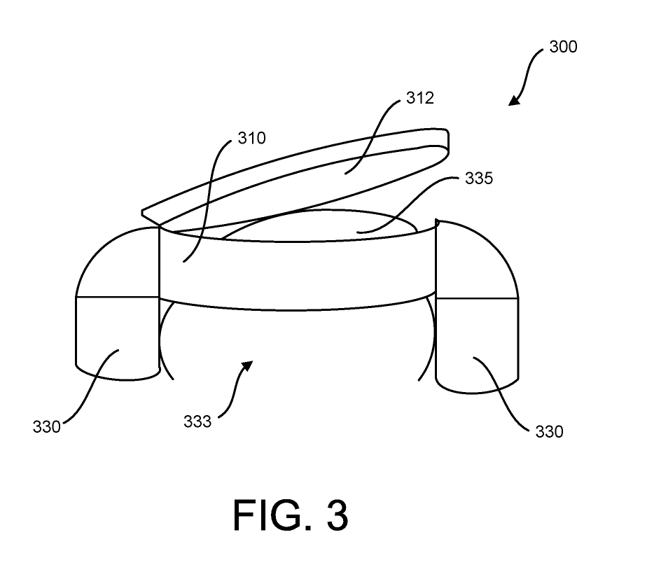

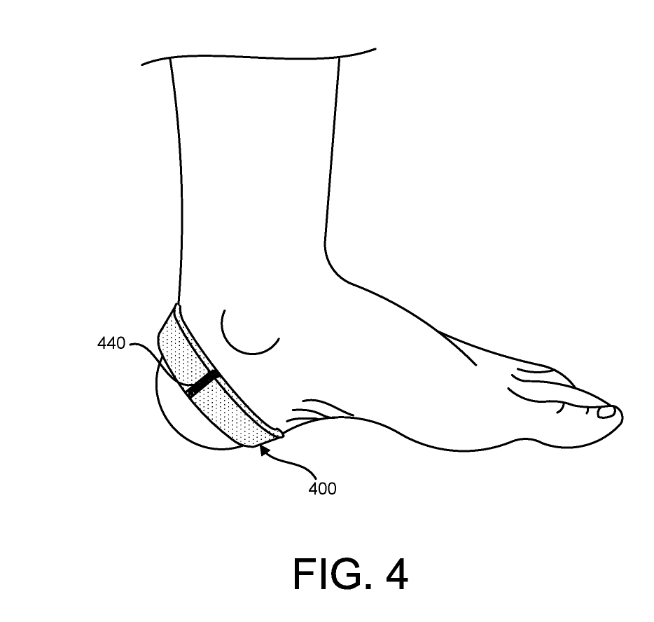

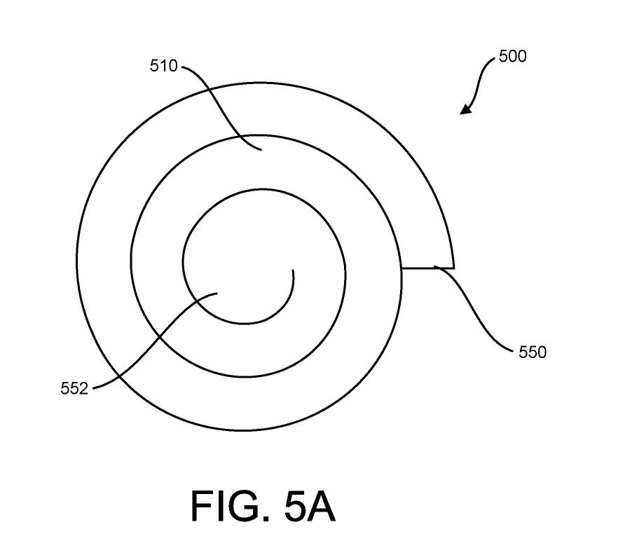

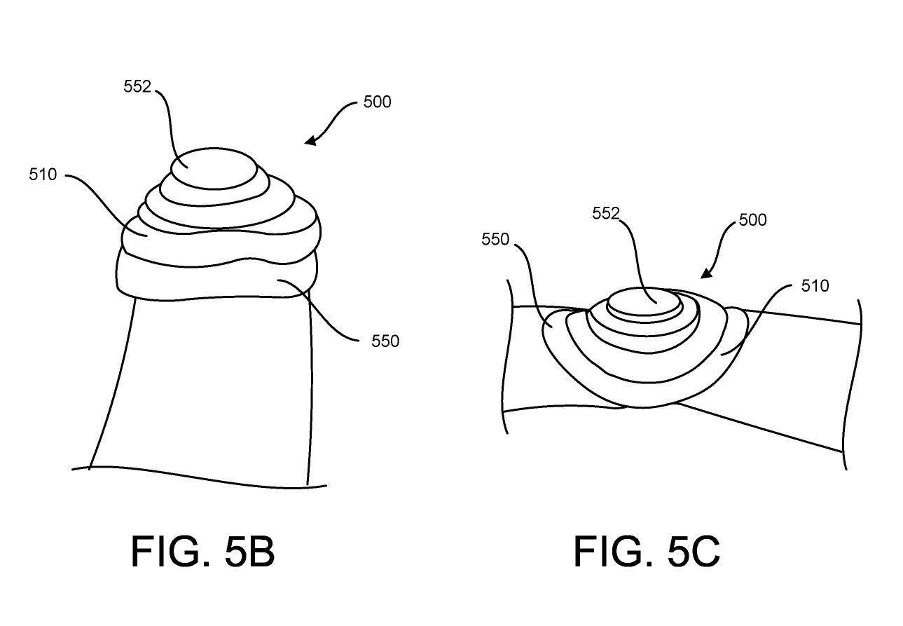

FIG. 5 illustrates another embodiment of the conformal thermal device 500 having a helical-shaped tubular body 510. As shown in FIG. 5, the helical-shaped tubular body 510 can include a first free end 550 and a second free end 552 with the second free end 552 being centrally positioned relative to the helical-shaped body 510 when in a coiled configuration. In some embodiments, the second free end 552 has a circular shape that can be beneficial for placing on a part of a joint, such as a knee cap of the knee joint, and then allowing the remaining length of the helical-shaped tubular body 510 to expand or conform around the joint, such as shown in FIGS. 5B and 5C.

FIGS. 5B and 5C illustrate the conformal thermal device 500 positioned along a bent knee (see FIG. 5B) and a straight knee (see FIG. 5C). In both configurations of the knee, the conformal thermal device 500 is able to conform to the knee joint. As such, the conformal thermal device 500 can provide effective and efficient thermal therapy to a joint (e.g., shoulder joint, knee joint, hip joint, etc.) regardless of the configuration of the joint. In some implementations, the body of the conformal thermal device includes a number of various sized rings that can be stacked on a body part to have a similar effect.

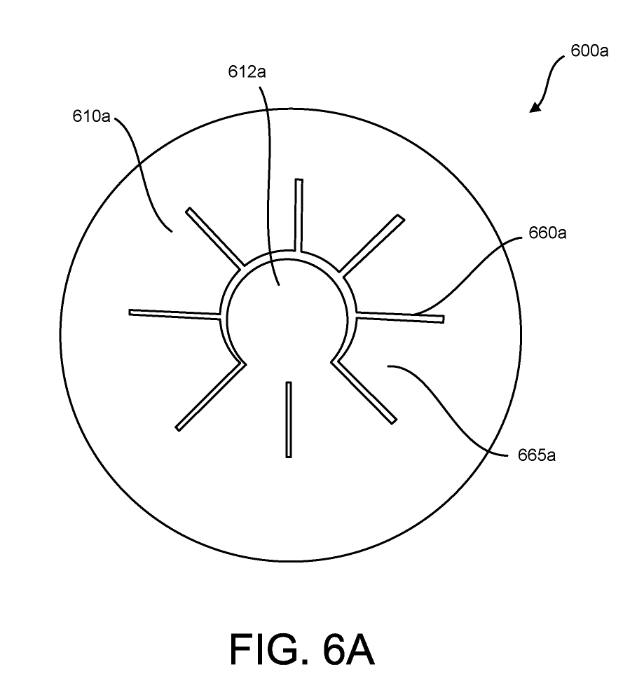

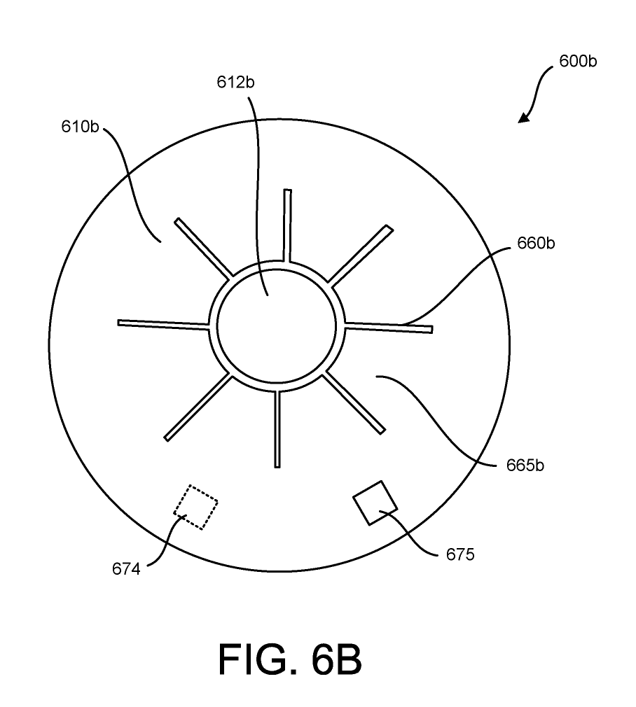

FIG. 6A illustrates another embodiment of the conformal thermal device 600a having a disc-shaped body 610a with a plurality of sub-chambers 665a that are in fluid communication with each other, including a center part 612a that includes an inner chamber or sub-chamber containing thermal material. The conformal thermal device 600a also includes one or more seams or cutouts 660a that define at least one of the sub-chambers 665a and the center part 612a. The seams and/or cutouts 660a can act similar to a joint where bending of the main body 610a can easily occur. In addition, such seams or cutouts 660a can allow the device 600a to effectively conform to a variety of body parts, including a knee joint where the center part 612a can be positioned on the knee cap of the user. For example, a seam 660a can include a heat-seal created between a top surface and a bottom surface of the body and a cutout 660a can include material that has been removed along the body 610a, thus creating a gap or passageway along the body. Each seam and/or the cutout can assist with defining one or more sub-chambers and can assist with allowing the body of the device to conform and collapse around a body part while keeping sufficient thermal material distributed about the body of the device (e.g., at least some material can remain in each sub-chamber).

FIG. 6B illustrates another embodiment of the conformal thermal device 600b having a plurality of sub-chambers 665b that are in fluid communication with each other (e.g., to allow thermal material to flow between sub-chambers 665b). The conformal thermal device 600b can include a center part 612b having an enclosed inner chamber filled with a thermal material. The conformal thermal device 600b can also include one or more seams or cutouts 660b that define at least one of the sub-chambers 665b and the center part 612b, such as described above for FIG. 6A. The conformal thermal device 600b can further include at least one indicator 675, such as either a time indicator or a thermal indicator, as shown in FIG. 6B. Each indicator can include at least one sensor. For example, the indicator 675 shown in FIG. 6B can be a thermal indicator and can include a temperature sensor for sensing either the contact surface of the user or the contact surface of the device. In some embodiments, the temperature indicator can include a temperature sensing strip that is embedded into an outer surface of the body of the device 600b. Although the indicator 675 is shown as being located in a single position along the body, each indicator 675 can include one or more parts (e.g., a first part that provides information to the user, such as the sensed temperature, and a second part that includes a sensor). For example, the conformal thermal device 600b can include a temperature indicator 675 that includes a temperature sensor positioned along a bottom surface of the body that is placed in contact with a body part of the user and an information window located along a top surface of the body that is viewable by the user during use, and which provides information to the user (e.g., sensed temperature readings, duration the device has been in contact with the body part, etc.). The indicator 675 can provide information in a variety of ways, such as those discussed above, for example, provide color changes to indicate temperature and/or time. As shown in FIG. 6B, the conformal thermal device 600b can include a connector 674 that is configured to releasably couple a modular extension to the body 610b. Such modular extension can include any size and shaped body further include one or more of the features described herein, including a complimenting connector that releasably couples to the connector 674. The connectors can include any of a variety of coupling mechanisms and features, such as Velcro, snaps, hooks, etc.

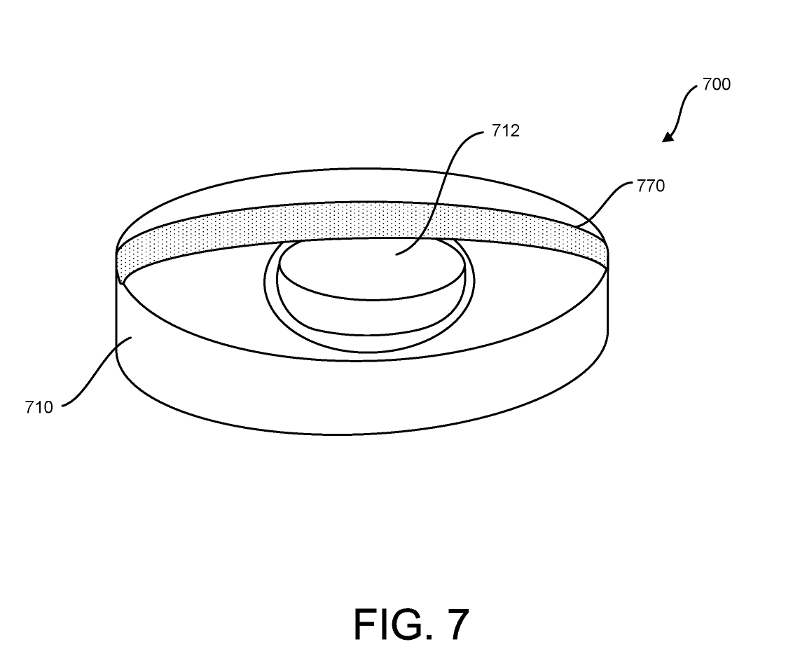

FIG. 7 illustrates another embodiment of the conformal thermal device 700 showing an adjustable coupling mechanism 770 coupled to and extending from the main body 710 of the device 700. As discussed above, the adjustable coupling mechanism can assist with securing the position of the device relative to one or more body parts while also allowing the body parts to move and function without (or at least limited) impedance from the device 700 and the coupling mechanisms 770. For example, the adjustable coupling mechanism 770 can be extended and shortened to provide a secure fit of the device 700 against a body part.

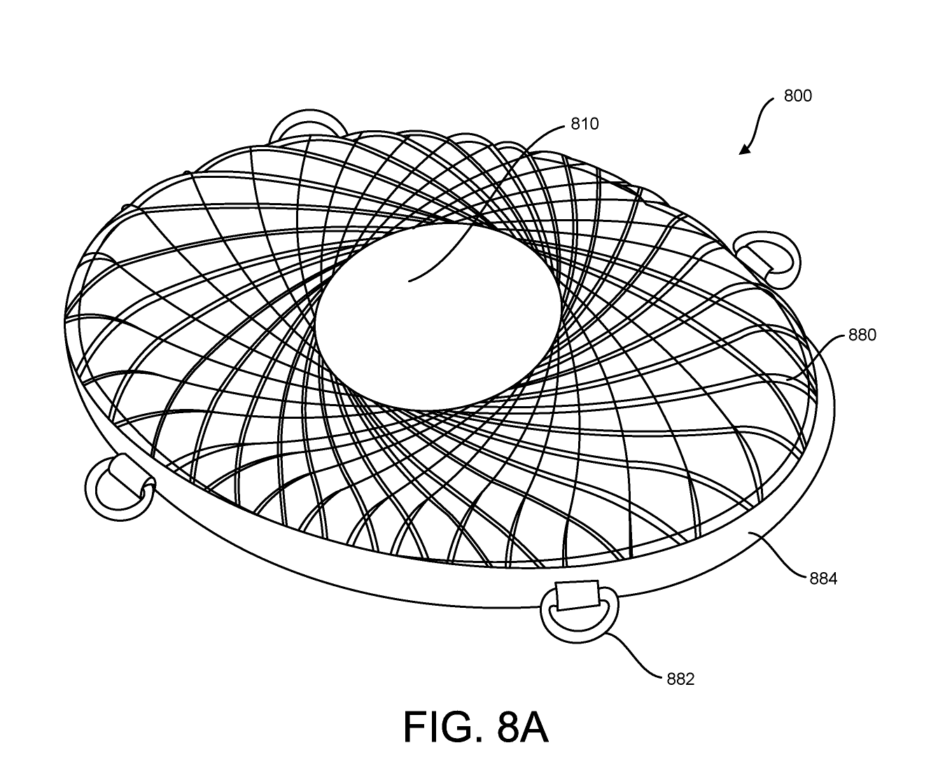

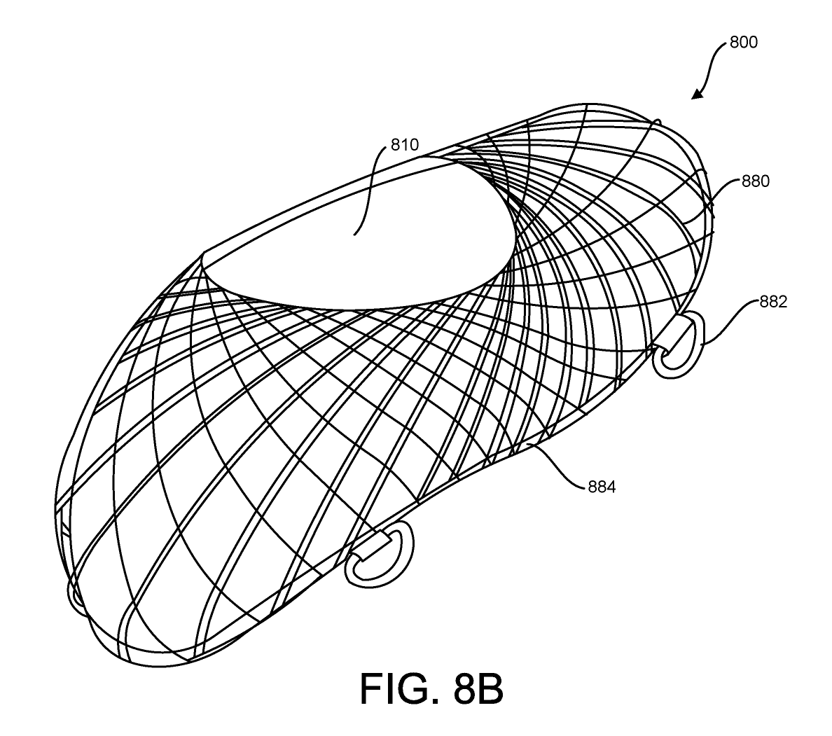

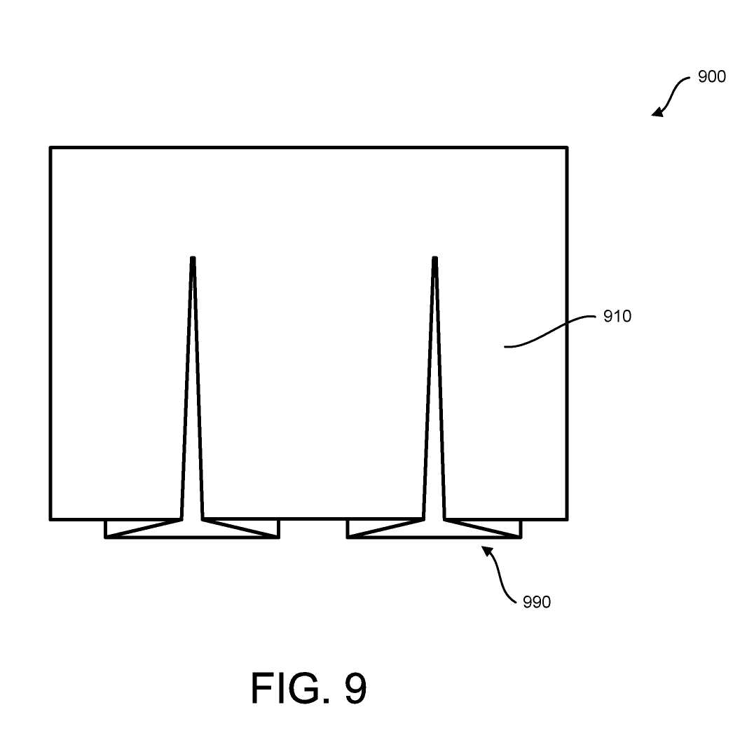

FIGS. 8A-8B show perspective views of a flexible cover 880 that is configured to cover an outer surface of the conformal thermal device 800 and provide either insulation or moisture wicking properties. For example, the cover 880 can include a flexible or deformable netting that can allow the body 810 of the conformal thermal device 800 to be inserted and removed from the cover 880. The cover 880 can also include a contact surface 884 that is configured to be placed against a body part of the user. The contact surface 884 can include insulation properties that prevent the passage of heat therethrough. This can be beneficial for directing heat from the body 810 to the body part in need of heating. In some embodiments, the contact surface 884 can include a waterproof or neoprene material that at least limits the passage of moisture therethrough. One or more coupling features 882 can be positioned around a perimeter of the cover 880 that can allow an adjustable coupling feature to be coupled thereto for assisting with securing the cover 880 and conformal thermal device 800 to the body part. Additionally or alternatively, the contact surface 884 can include reflective properties that can assist with preventing the transfer of heart in an undesired direction (e.g., away from the body part if heating the body part or towards the body part if cooling the body part). In some embodiments, the contact surface 884 can include a moisture-wicking property that can collect moisture present along and adjacent the body 810 of the device 800.

FIG. 10 illustrates another embodiment of the conformal thermal device 900 that includes one or more folded pleats 990 formed along the main body 910. Such pleats 990 can allow the device 900 to expand and collapse around one or more body parts for providing heating or cooling.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed is:

1. A conformal thermal device, comprising:
an outer skin that defines a body of the conformal thermal device, the outer skin being flexible and configured to conform to a body part of a user;
an inner chamber defined by and enclosed within the outer skin; and
a thermal material that is configured to provide cooling or heating to the body part, the thermal material being contained within the inner chamber.

2. The conformal thermal device of claim 1, wherein the body includes one or more of a ring shape, a helical shape, and a torus shape.

3. The conformal thermal device of claim 1, wherein the body further includes one or more cutouts that extend through a disc-shaped body and are radially positioned about a center of the disc-shaped body.

4. The conformal thermal device of claim 1, further comprising an adjustable coupling mechanism that extends from the outer skin and is configured to releasably couple the body of the conformal thermal device to the body part such that the body part is able to perform unimpeded movements.

5. The conformal thermal device of claim 1, wherein the body includes one or more heat seals that define at least two sub chambers that are in fluid communication with each other and allow the thermal material to flow therebetween.

6. The conformal thermal device of claim 1, further comprising one or more of an elastic support and a compressible support positioned within the body and configured to expand and/or compress in response to movement of the body part.

7. The conformal thermal device of claim 1, further comprising a first indicator that is configured to indicate when at least one of the thermal material, an outer surface of the outer skin, and a top surface of the user is at a temperature within a defined temperature range

8. The conformal thermal device of claim 7, wherein the first indicator is configured to change color to inform the user that the temperature is within the desired temperature range.

9. The conformal thermal device of claim 7, wherein the defined temperature range includes temperatures of approximately 0 degrees Celsius to approximately 10 degrees Celsius or approximately 18 degrees Celsius to approximately 38 degrees Celsius.

10. The conformal thermal device of claim 1, further comprising a second indicator that is configured to inform the user when the outer skin has been conformed to the body part for a duration that is within a defined time range.

11. The conformal thermal device of claim 10, wherein the second indicator is configured to change color to inform the user that the duration is within the defined time range.

12. The conformal thermal device of claim 10, wherein the defined time range includes approximately 18 minutes to approximately 25 minutes.

13. The conformal thermal device of claim 1, further comprising an adjustable weight that is at least one of coupled to and integrated with the outer skin, wherein the adjustable weight is configured to provide adjustable compression between the outer skin and the body part.

14. The conformal thermal device of claim 1, wherein the body is pleated.

15. The conformal thermal device of claim 1, further comprising a cover that is configured to extend around a part of the body and includes an insulation material that limits the release of heat away from the body part.

16. The conformal thermal device of claim 1, further comprising a cover that is configured to extend around a part of the body and includes a moisture-wicking material that collects moisture on and adjacent to the body.

17. The conformal thermal device of claim 1, further comprising at least one modular extension that includes thermal material and is configured to provide cooling or heating, each of the modular extensions being configured to releasably couple to the body of the conformal thermal device.

18. The conformal thermal device of claim 17, further comprising at least one connector along the outer skin, each of the connectors being configured to releasably couple one or more of the least one modular extension.

19. A method comprising:
conforming an outer skin of a conformal thermal device against a body part of a user, the conformal thermal device including a thermal material that is configured to provide heating or cooling to the body part; and
providing heating or cooling to the body part.
